(19) United States
(12) Patent Application Publication
(10) Pub. No.: US 2019/0330140 A1
(43) Pub. Date: Oct. 31, 2019

(54) Compound for Use in Enzymatic Reaction and Mass Spectrometry Method

(71) Applicant: HITACHI HIGH-TECHNOLOGIES CORPORATION, Tokyo (JP)

(72) Inventors: Hiroaki NAKAGAWA, Tokyo (JP); Masahiko YOSHIDA, Tokyo (JP)

(73) Assignee: Hitachi High-Technologies Corporation, Tokyo (JP)

(21) Appl. No.: 16/312,787

(22) PCT Filed: Jun. 22, 2017
(86) PCT No.: PCT/JP2017/022994

§ 371 Date: Dec. 21, 2018

(30) Foreign Application Priority Data
Jun. 29, 2016 (JP) 2016-128294

(51) Int. Cl. C07C 233/15; G01N 27/62; C07H 15/203

(52) U.S. Cl. CPC C07C 233/15; G01N 2400/32; C07H 15/203; G01N 27/622

(57) ABSTRACT

A compound used in the conventional enzymatic reactions and mass spectrometry methods needs to be altered with respect to the structure thereof as a substrate compound, such as the length of an alkyl chain contained therein, depending on the type of a target enzyme, and therefore has the problem that the conditions for the mass spectrometry on a product compound are undesirably varied and the sensitivity is deteriorated. In the present invention, a compound is provided, which can be used in an enzymatic reaction and a microanalysis method both for detecting a trace component stably and with high sensitivity. The compound according to the present invention is characterized by having a nitrogen atom, an amide bond and a glycosidic bond at specific sites, respectively, has high reactivity with an enzyme, and can provide a compound capable of being detected very easily with a mass spectrometer.

### TECHNICAL FIELD

The present invention relates to a compound that can be used in an enzymatic reaction and a mass spectrometry method both for detecting a trace component, and also to an enzymatic reaction and a mass spectrometry method that use the compound.

### BACKGROUND ART

A mass spectrometer is a device that ionizes a substance and measures the m/z (a value obtained by dividing the mass of an ion by the unified atomic mass unit and by the charge number of the ion, written in italicized letters) and the intensity on the basis of the ion mobility in vacuum. Although control is performed to introduce only ions having a specific m/z value into a detector, the m/z values of the introduced ions have a certain range and different ion species may have the same m/z. In order to more specifically select an ion species, fragment ions generated by cleavage are utilized. First, ions are subjected to primary selection by m/z, and then the ions are cleaved, and the fragment ions generated by the cleavage of the bonds within the ions are subjected to secondary selection by m/z. The selectivity can thus be improved. The selected reaction monitoring (SRM) in which specific fragment ions generated by the cleavage are continuously detected is an analysis method having high selectivity and quantitativity. In this technique, the sensitivity and reproducibility are higher when the specific fragment ions are generated more stably and the production yield is higher. Organic compounds have a main chain, as a backbone of the structure, in which carbon atoms are linked together, and when a nitrogen atom or oxygen atom is contained in the main chain, such an organic compound is likely to be cleaved at a specific position (NPL 1).

Amplification of a compound to be detected using an enzyme has been widely carried out in analyses of biocomponents. For example, a galactosidase gene may be incorporated together with a target gene to confirm whether a specific gene is expressed in a gene recombination. A galactosidase is expressed at the same time as expression of a specific gene, and if 5-bromo-4-chloro-3-indolyl β-D-galactopyranoside is added, galactose is released from the compound by an enzymatic reaction to give a color. Thus, the expression of the specific gene can be determined (NPL 2).

It is a known technique in an enzyme immunization to optically detect a compound that is produced by a reaction of an enzyme bound to an antibody or an enzyme bound to an antibody via a biotin-avidin composite. In a known system, for example, a peroxidase is used as an enzyme, and tetramethylbenzene which is a reaction substrate receives two electrons to form a quinoniminium double cation radical which gives a blue color. Such methods all take advantage of the fact that an enzymatic reaction, which allows multiple molecules of a substrate to react with one molecule of an enzyme, amplifies the substrate (NPL 3).

As a method for detecting an enzymatic reaction by detecting a product compound produced by a reaction of a substrate compound with an enzyme by using a mass spectrometry, a method using a lysosome enzyme and a substrate that targets the lysosome enzyme is known (PTL 1).

In such methods, the structure of the substrate compound, such as the length of an alkyl chain contained therein, needs to be altered depending on the type of a target enzyme, and therefore there are problems in that the conditions for the mass spectrometry on a product compound are undesirably varied and the sensitivity is deteriorated. Thus, the methods have not been satisfactory as a method for stable microanalysis of an analysis target.

### CITATION LIST

### Patent Literature

PTL 1: JP-T-2009-530310 (the term “JP-T” as used herein means a published Japanese translation of a PCT patent application.)

### Non-Patent Literature

NPL 1: Hisao Nakata, J. Mass Spectrom. Soc. Jpn., 63, 1, 31-43, 2015
NPL 2: S. Muto, Q-M. Zhang, S. Yonei, J. Bacteriol., 175, 2645-2651, 1993
NPL 3: The Immunoassay Handbook Fourth Edition, edited by David Wild, 2013, Elsevier Publishing (the UK)

### SUMMARY OF INVENTION

### Technical Problem

Thus, an object of the present invention is to provide a compound that can be used in an enzymatic reaction and a microanalysis method both for detecting a trace component stably and with high sensitivity, and an enzymatic reaction and a mass spectrometry method that use the compound.

### Solution to Problem

In intensive studies of compounds that are suited for an enzymatic reaction and mass spectrometry in view of the above abject, the present inventors have found that a substrate compound that has an nitrogen atom, an amide bond, and a glycosidic bond at specific sites, respectively, has high reactivity with an enzyme, and that a compound (product compound) produced by the enzymatic reaction can be detected very easily by a mass spectrometer, completing the present invention.

As used herein, the phrase “can be detected easily” means that a high signal intensity is provided in a mass spectrometer as well as that the difference between the mass of a product compound which is a detection target and the mass of a substrate compound is so large that a result of mass spectrometry of the product compound does not interfere with a result of the mass spectrometry of the substrate compound. In mass spectrometry, compounds to be detected are separated by chromatography in many cases, and the above phrase means that the separation is easy. In such a separation, a separating agent that mainly recognizes the hydrophobicity of compounds is often used, and a large difference is required between the hydrophobicity of the substrate compound and that of the product compound.

Specifically, the present invention relates to the followings.

[1] A compound represented by the general formula (1):

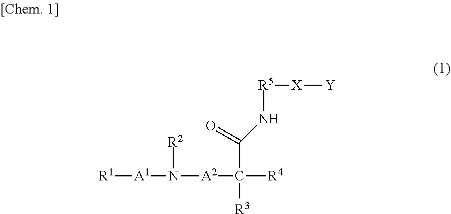

or the general formula (2):

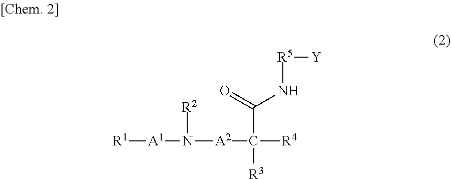

or the general formula (3):

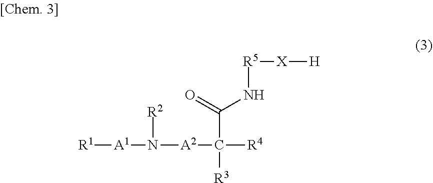

or the general formula (4):

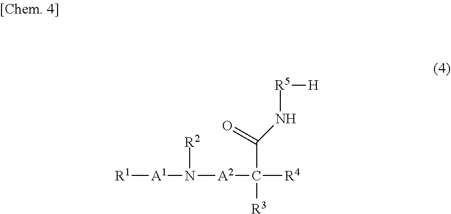

(in the general formulae (1), (2), (3), and (4), R¹, R², R³, and R⁴ may be the same as or different from each other and each represent an alkyl group, an aryl group, a cycloalkyl group, or a heterocyclic group having no substituent or having a substituent W, W represents a C1 to 10 saturated or unsaturated hydrocarbon group, an aryl group, a heterocyclyl group, an alkoxy group, a fluoroalkyl group, an acyl group, an ester group, a hydroxyl group, an amino group, an amide group, a carboxyl group, a sulfonyl group, a nitro group, a cyano group, a sulfenyl group, a sulfo group, a mercapto group, a silyl group, or a halogen group, R5 represents an aryl group, a cycloalkyl group, or a heterocyclic group not having a substituent other than an —X—Y group, a —Y group, and an —X—H group, or having a substituent W other than an —X—Y group, a —Y group, and an —X—H group, A¹ and A² may be the same as or different from each other and each represent an alkyl group having no substituent or having a substituent W, and X represents a sulfur atom or an oxygen atom, and Y in the general formulae (1) and (2) represents a saccharide), or a salt thereof.

[2] The compound according to [1], wherein X in the general formulae (1) and (3) is an oxygen atom, and wherein the compound has a octanol/water partition coefficient, Log P, of 1 to 5, and has a molecular weight of 100 to 1000, and a salt thereof.

[3] A mass spectrometry method, including a step of reacting an enzyme with the compound of the general formula (1) or (2) according to claim 1 to obtain the compound of the general formula (3) or (4) according to [1] or [2].

[4] The mass spectrometry method according to [3], wherein the enzyme is a glycosidase.

### Advantageous Effects of Invention

According to the present invention, it is possible to detect a trace of an enzyme or the like used in an enzymatic reaction in an enzyme immunization or the like stably and with high sensitivity using a mass spectrometer.

### BRIEF DESCRIPTION OF DRAWINGS

FIG. 1 It is a view illustrating HPLC data regarding the compound HV.

FIG. 2 It is a view illustrating NMR data regarding the compound HV.

FIG. 3 It is a view illustrating HPLC data regarding the compound HVG.

FIG. 4 It is a view illustrating NMR data regarding the compound HVG.

FIG. 5 It is a view illustrating an analytical result of 3.0 pg/mL of a sample of the compound HVG.

FIG. 6 It is a view illustrating an analytical result of 3.0 pg/mL of a sample of the compound HV.

### DESCRIPTION OF EMBODIMENTS

The substrate compound of the present invention suitable for an enzymatic reaction and mass spectrometry is represented by the following general formula (1) or (2), and the product compound is represented by the following general formula (3) or (4). The compound represented by the following general formula (1) or (2) reacts with an enzyme and the compound represented by the following general formula (3) or (4) which is a product compound is then analyzed with a mass spectrometer, thereby making it possible to detect a specific enzyme stably and with high sensitivity:

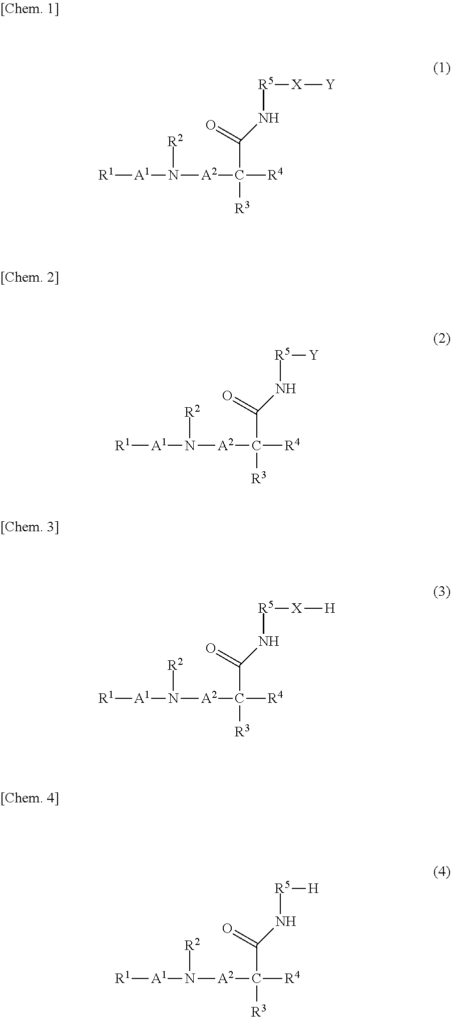

In the general formulae (1), (2), (3), and (4), R¹, R², R³, and R⁴ may be the same as or different from each other and each represent an alkyl group, an aryl group, a cycloalkyl group, or a heterocyclic group having no substituent or having a substituent W. Examples of alkyl groups include a methyl group, an ethyl group, an n-propyl group, an isopropyl group, an n-butyl group, a sec-butyl group, a tert-butyl group, and other alkyl groups having 1 to 10 carbon atoms. Examples of aryl groups include a phenyl group and a naphthyl group. Examples of cycloalkyl groups include a cyclopentyl group and a cyclohexyl group. Examples of heterocycles include an imidazole ring, an imidazoline ring, an imidazolidine ring, a 1,2,4-triazole ring, a tetrazole ring, an oxazoline ring, an oxazole ring, an oxazolidine ring, a thiazoline ring, a triazole ring, and a thiazolidine ring. Examples of substituents W include a C1 to 10 saturated or unsaturated hydrocarbon group, an aryl group, a heterocyclyl group, an alkoxy group, a fluoroalkyl group, an acyl group, an ester group, a hydroxyl group, an amino group, an amide group, a carboxyl group, a sulfonyl group, a nitro group, a cyano group, a sulfenyl group, a sulfo group, a mercapto group, a silyl group, and a halogen group.

In the general formula (1), (2), (3), and (4), R5 represents an aryl group, a cycloalkyl group, or a heterocyclic group having no substituent other than an —X—Y group, a —Y group, and an —X—H group or having a substituent W other than an —X—Y group, a —Y group, and an —X—H group, A¹ and A² may be the same as or different from each other and each represent an alkyl group having no substituent or having a substituent W, and X is a sulfur atom or an oxygen atom. Examples of alkyl groups include a methyl group, an ethyl group, an n-propyl group, an isopropyl group, an n-butyl group, a sec-butyl group, a tert-butyl group, and other alkyl groups having 1 to 10 carbon atoms. Examples of aryl groups include a phenyl group and a naphthyl group. Examples of cycloalkyl groups include a cyclopentyl group and a cyclohexyl group. Examples of heterocycles include an imidazole ring, an imidazoline ring, an imidazolidine ring, a 1,2,4-triazole ring, a tetrazole ring, an oxazoline ring, an oxazole ring, an oxazolidine ring, a thiazoline ring, a triazole ring, and a thiazolidine ring. Examples of substituents W include a C1 to 10 saturated or unsaturated hydrocarbon group, an aryl group, a heterocyclyl group, an alkoxy group, a fluoroalkyl group, an acyl group, an ester group, a hydroxyl group, an amino group, an amide group, a carboxyl group, a sulfonyl group, a nitro group, a cyano group, a sulfenyl group, a sulfo group, a mercapto group, a silyl group, and a halogen group.

In the general formula (1) and (2), Y is a saccharide, and a carbon atom at position 1 in the saccharide binds to R⁵ or X. Examples of saccharides include pentoses and hexoses. Examples of hexoses include aldohexoses and ketohexoses. Examples of aldohexoses include galactose, glucose, and mannose, and examples of ketohexoses include fructose, psicose, and sorbose. Among saccharides, aldohexoses are preferred from the viewpoint of availability of an enzyme having high substrate specificity and high activity, and among them, glucose and galactose are more preferred and D-glucose and D-galactose are further preferred.

Examples of salts of the compounds represented by the general formulae (1), (2), (3), and (4) include hydrochlorides, nitrates, sulfates, and acetates thereof. From the viewpoint of production and solubility, hydrochlorides are preferred.

Examples of enzymes used in the enzymatic reaction include glycosidases. Examples of glycosidases include galactosidases, glucosidases, monnosidases, chitinases, fucosidases, amylases, isoamylases, cellulases, lactases, and hexosaminidases. From the viewpoint of activity and specificity, a β-galactosidase containing a lactase and an α-glucosidase containing an amylase are preferred.

Regarding the compounds represented by the general formulae (1) and (2), a feature that the compound has a group having a nitrogen atom, an amide bond, and a glycosidic bond at specific sites, respectively, and the R⁵ moiety is an aryl group, a cycloalkyl group, or a heterocyclic group provides a characteristic that the enzymatic reaction proceeds well. For example, for facilitating the release of the —Y group in the general formula (1) or (2) by an enzymatic reaction, the R⁵ moiety is preferably an aryl group, a cycloalkyl group, or a heterocyclic group having a 6-membered structure.

Regarding the compounds represented by the general formulae (1), (2), (3), and (4), a feather that the compound has an amide bond at a specific site provides a characteristic that cationic ions are likely to be produced in ionization in a mass spectrometry measurement, leading to an effect of increasing the sensitivity in measurements by mass spectrometers.

Regarding the compounds represented by the general formulae (1) and (2), a feature that the compound has a glucosidic bond at a specific site provides a characteristic that the selectivity in cutting by a specific enzyme is enhanced, leading to an effect that the compound of (3) or (4) is produced only in the presence of a specific enzyme.

Regarding the compounds represented by the general formulae (3) and (4), the compound preferably has a nature of easily dissolving in a buffer-organic solvent system for providing a strong signal intensity in a mass spectrometer, and specifically the octanol/water partition coefficient (log P) is preferably in the range of 1 to 5.

Compound represented by the general formulae (3) and (4), which have a group that has a nitrogen atom at a specific site in the carbon-carbon bonds in the main chain and further have an amide bond at a specific site, leads to generation of specific fragment ions, enabling the stable and highly sensitive detection.

Compounds having different masses sometimes interfere with each other in mass spectrometry due to the presence of an isotope or an adduct ion. Known examples of adduct ions include ions with a hydrogen atom, ammonium, sodium, and potassium. For eliminating the interference, the m/z value (mass/charge number) of the substrate compound and that of the product compound are preferably different by 40 or more.

Examples of contaminants include compounds derived from the sample as well as water or an organic solvent, such as acetonitrile, used in a mobile phase, salts with ammonia, formic acid, and the like, and a cluster of few molecules thereof. For avoiding the interference with such fragment ions, compounds represented by the general formula (1) and the general formula (2) preferably have m/z values of 100 or more. For example, when Y in the general formula (1) is D-galactose and β-galactosidase is used as an enzyme, the difference of the molecular weights of the substrate compound and the product compound is 162 since the β-galactosidase releases the galactose, and the compounds can be clearly distinguished in a mass spectrometer.

A substrate compound and a product compound are generally both present in an enzymatic reaction solution, and it is assumed that the substrate compound inhibits the ionization of the product compound. For avoiding this, the compounds are separated by chromatography. For facilitating the separation between a substrate compound and a product compound by chromatography, a product compound that has a significantly different structure and nature from those of the substrate compound is preferably produced in the enzymatic reaction. For example, a separating agent that mainly recognizes the hydrophobicity of a compound is often used in chromatography. Accordingly, a substrate compound and a product compound are preferably significantly different in the hydrophobicity.

In the present invention, a saccharide, which is a hydrophilic compound, is separated by an enzymatic reaction, and therefore the substrate compound and the product compound are significantly different in the hydrophobicity. For example, when the substrate compound represented by the general formula (1) or (2) is a compound of [Chem. 5] shown below (hereinafter referred to as “HVG”), the calculated value of the log P is 3.6, and by separating highly hydrophilic galactose (log P value −3.4) by an enzymatic reaction, a compound [Chem. 6] (hereinafter referred to as “HV”) shown below is produced as the compound represented by the general formula (3) or (4). The calculated value of log P of HV is 4.9 and thus has significantly different hydrophilicity as compared with the calculated value of log P of HVG.

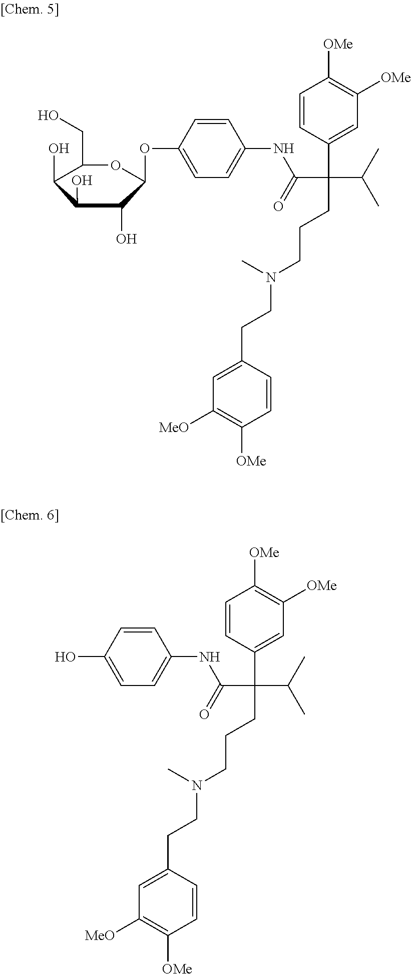

### EXAMPLES

The present invention will be more specifically described with reference to examples, but the present invention is not to be limited to the examples.

Products were analyzed in the conditions as described below.

Nucleic magnetic resonance apparatus (NMR)

VARIAN NMR Apparatus 400 MHz

Liquid chromatography mass spectrometer (LC-MS)

LC part conditions:
Apparatus: Shimadzu Corporation LC-20
Column: Agilent Technologies Eclipse XDB-C18
   (Column length 150 mm, inner diameter 4.6 mm, filler particle size 5 μm)
Column temperature: 40° C.
Flow rate: 1.5 mL/min
Detection device: Ultraviolet spectroscopy (UV) detector, measurement wavelength 254 nm
Eluent A: 0.037% (V/V) trifluoroacetic acid aqueous solution
Eluent B: 0.018% (V/V) trifluoroacetic acid acetonitrile solution

Gradient conditions: 0 min, Eluent B 10%
10 min, Eluent B 80%
15 min, Eluent B 80%

MS part conditions:
Shimadzu Corporation 2010 MSD (ionizer ES-API)

[Synthesis of Compound HV]

### Example 1

HV was synthesized by the following method.

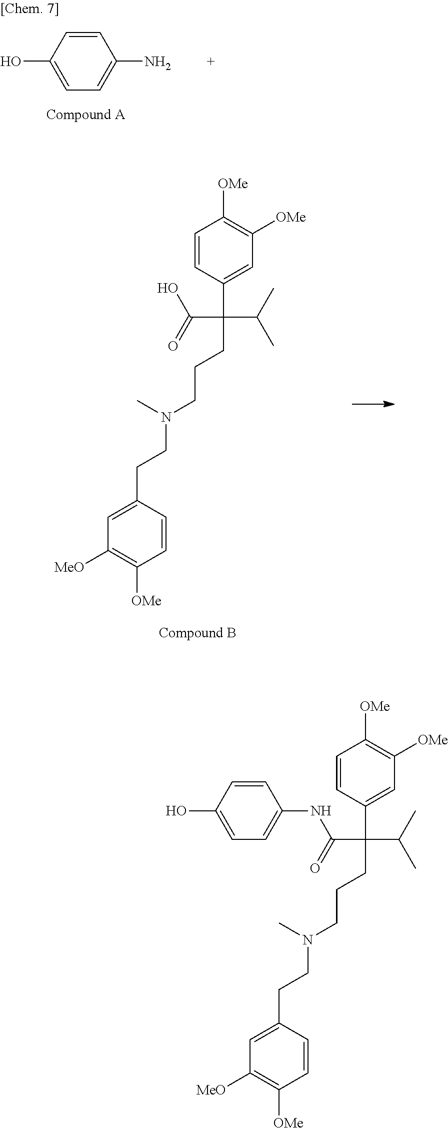 [SMILES: Nc1ccc(O)cc1 ; COc1ccc(CCN(C)CCCC(C(=O)O)(c2ccc(OC)c(OC)c2)C(C)C)cc1OC ; COc1ccc(CCN(C)CCCC(C(=O)Nc2ccc(O)cc2)(c2ccc(OC)c(OC)c2)C(C)C)cc1OC]

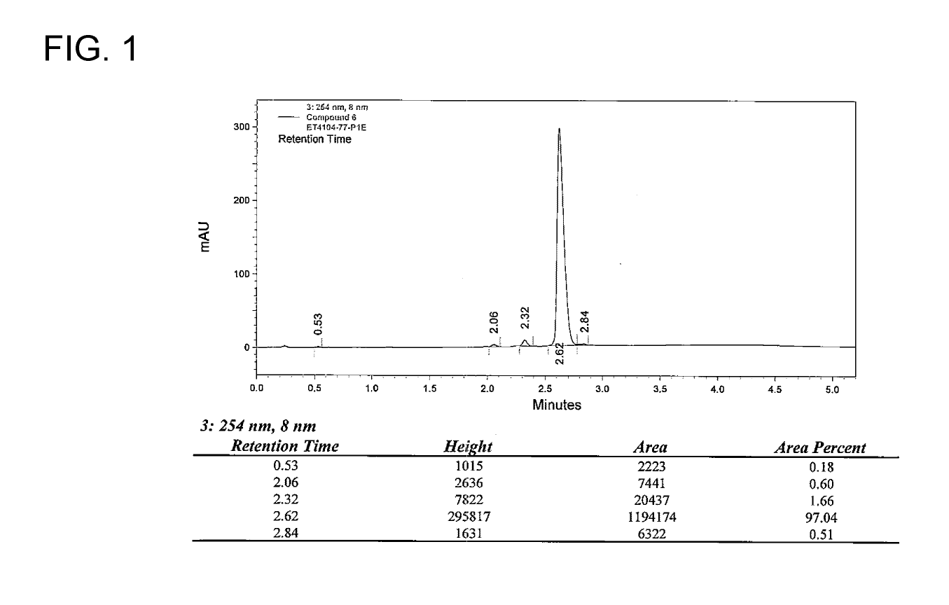

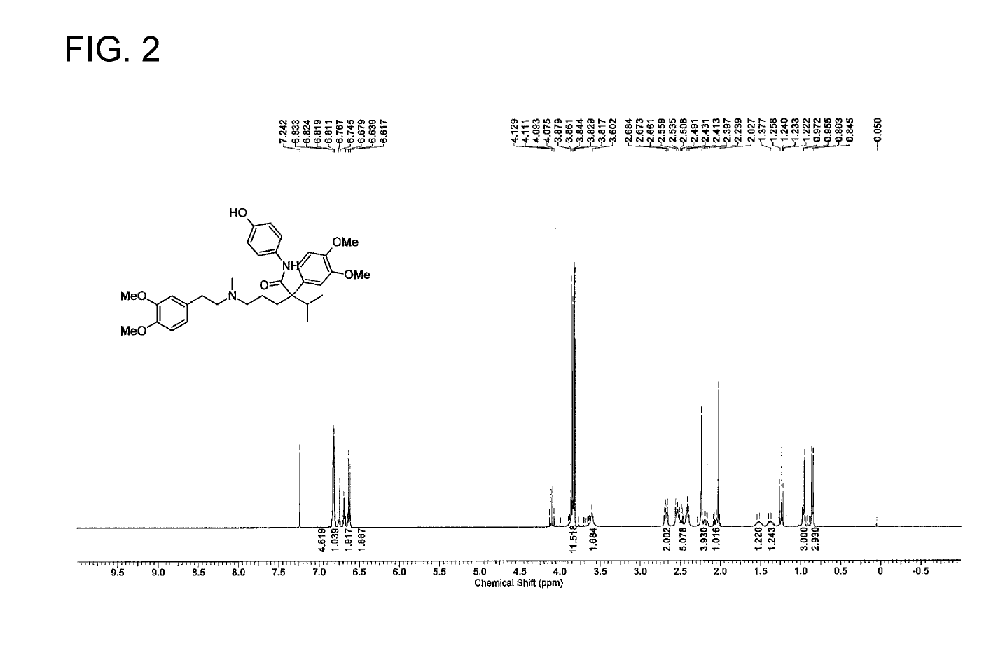

In a flask purged with nitrogen, a compound B (2.0 g, 4.2 mmol), 4-aminophenol as a compound A (1.15 g, 10.6 mmol, 2.5 equivalents), and 5 mL of DMF (N, N-dimethylformamide) as a solvent were put and cooled to 0° C. DMAP (N, N-dimethyl-4-aminopyridine, 115 mg, 1.3 mmol, 0.3 equivalents) and EDC.HCl (1-(3-dimethylaminopropyl)-3-ethylcarbodiimide hydrochloride, 890 mg, 4.7 mmol, 1.1 equivalents) were added thereto, and DIEA (N, N-diisopropylethylamine, 546 mg, 4.2 mmol, 1.0 equivalent) was added dropwise while keeping 0° C. The mixture was heated to 20° C. and was reacted for 3 hours. The reaction solution was poured into 150 mL of water, and was extracted five times with 150 mL of ethyl acetate, and the remaining aqueous phase was further extracted six times with 50 mL of a dichloromethane/methanol 10:1 mixture. All the obtained organic phases were combined and this liquid was washed three times with 50 mL of a saturated saline solution. The organic solvent was removed at 45° C. under reduced pressure. The resulting residue was purified by preparative HPLC (high performance liquid chromatography) under the following conditions to thereby obtain 0.6 g (25% yield) of the target HV. NMR supported the HV structure, and the HPLC measurement confirmed a purity of 97.0%. FIG. 1 illustrates the HPLC data and FIG. 2 illustrates the NMR data.

[Synthesis of Compound HVG]

### Example 2

HVG was synthesized by the following method.

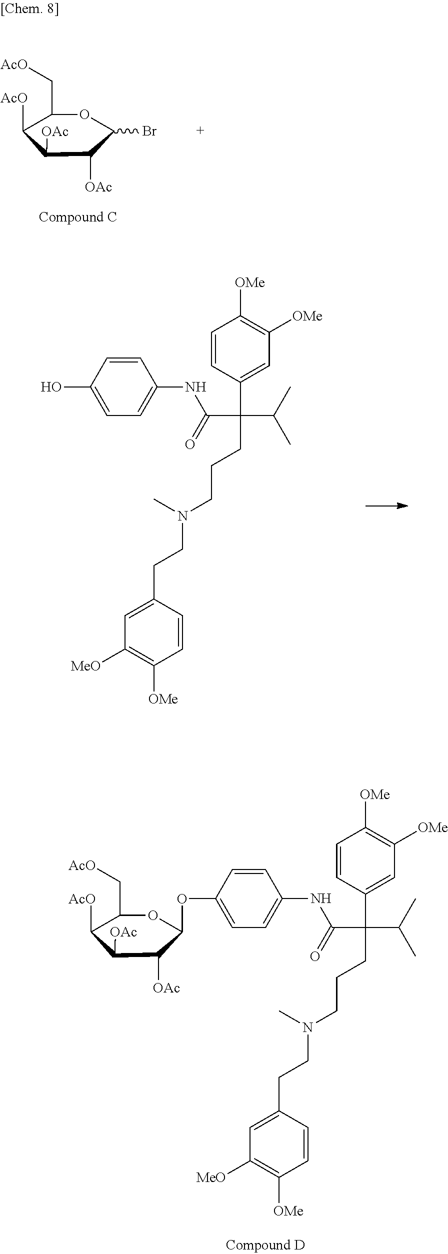

HV (50 mg, 88.5 μmol) and a compound C (109 mg, 226 μmol, 3 equivalents) were dissolved in 2.0 mL of DMF, cesium carbonate (115 mg, 354 μmol, 4 equivalents) was added at 20° C., and the mixture was reacted with stirring at the same temperature for 16 hours. The reaction mixture was poured into 20 mL of water and was extracted three times with 20 mL of ethyl acetate. All of the ethyl acetate extracts were combined and washed three times with 20 mL of a saturated saline solution, then dried over anhydrous sodium sulfate, and the solvent was removed at 40° C. under reduced pressure. The residue was purified by preparative TLC (silica gel, a dichloromethane and methanol mixture was used as an eluent) to thereby obtain 40 mg (38% yield) of HVA in the form of pale yellow oil as a compound D.

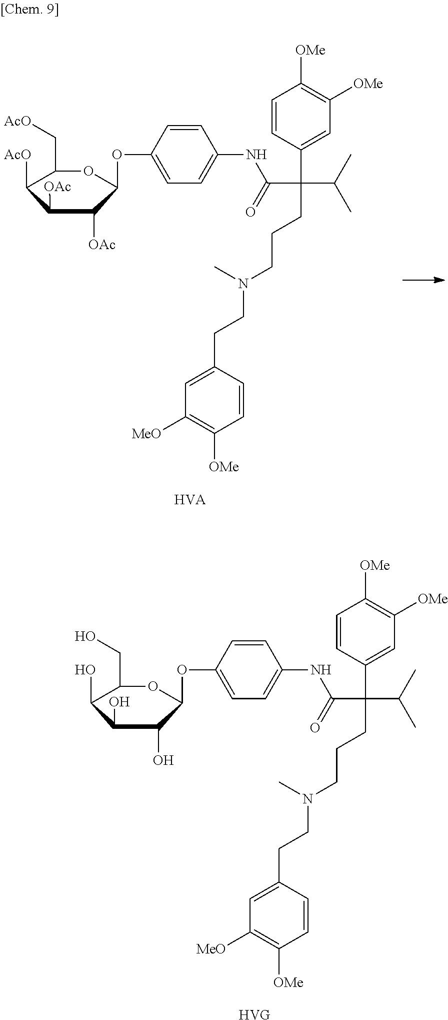

Under nitrogen stream, HVA (40 mg, 44.7 μmol) was added at a time to a 1 mol/L sodium methoxide methanol solution (8.94 μL, 0.2 equivalents) at 20° C., and the mixture was reacted at 25° C. for 3 hours. The reaction solution was distilled under reduced pressure and the residue was purified by preparative HPLC (the purification conditions are described below) to thereby obtain 3.3 mg (10% yield) of HVG in the form of pale yellow crystals.

Preparative HPLC Conditions

Apparatus: Gilson 281 semi-preparative HPLC system

Preparative column: Agela Venusil XBP C18
(Column length 150 mm, inner diameter 25 mm, filler particle size 5 μm)

Flow rate: 25 mL/min

Detection device: UV (ultraviolet visible spectroscopy) detector, measurement wavelengths 220 and 254 nm

Eluent A: 10 mM ammonium hydrogen carbonate aqueous solution

Eluent B: acetonitrile

Gradient conditions: 0 min, Eluent B 20%
12 min, Eluent B 40%
14 min, Eluent B 100%

NMR and LC-MS (calculated value of molecular weight 726) supported the HVG structure, and the HPLC measurement (column:

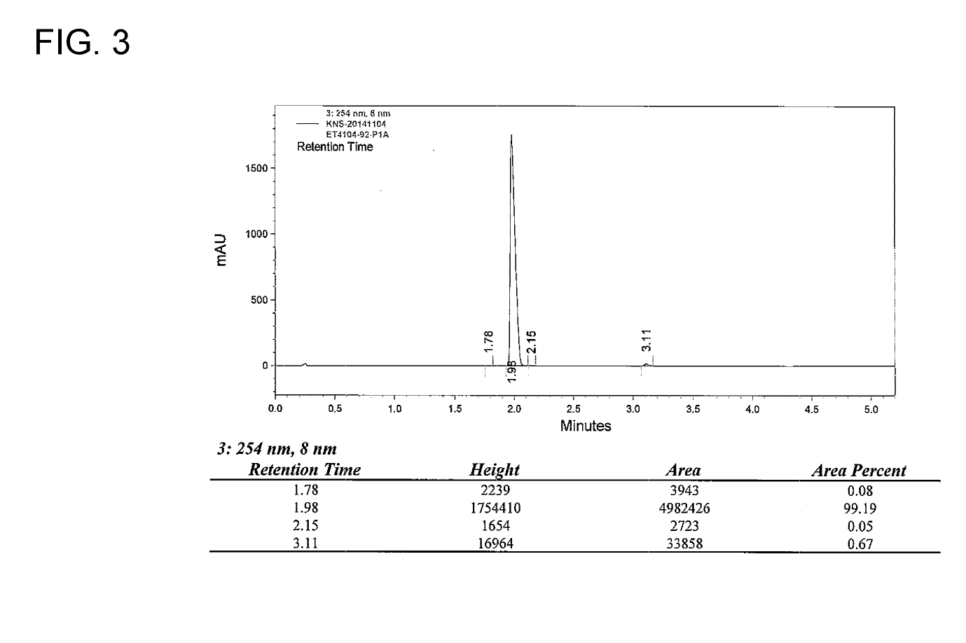

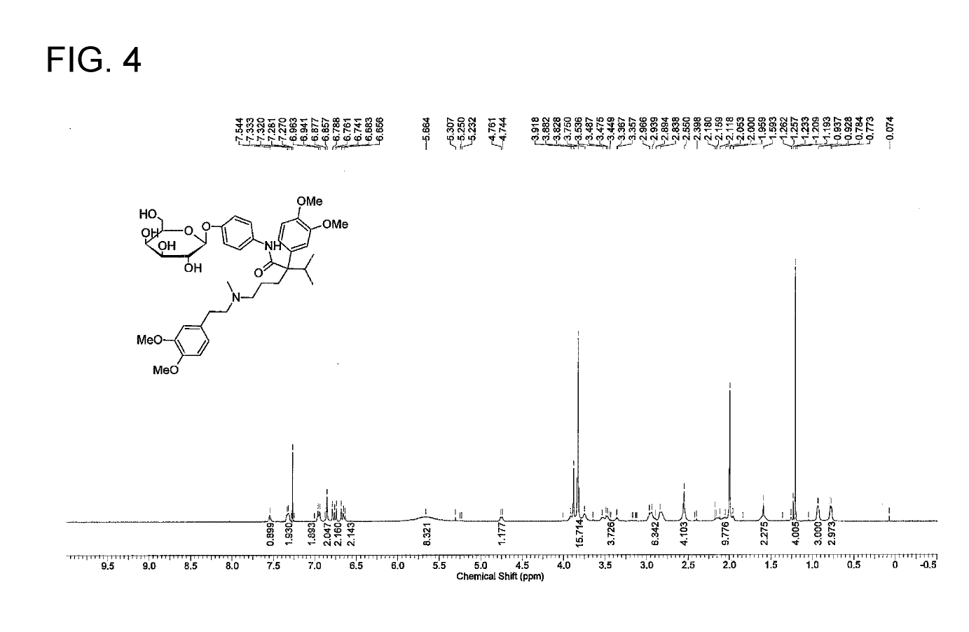

Phenomenex LUNA C18 column length 50 mm, inner diameter 2 mm, filler particle size 5 μm) confirmed a purity of 99.2%. FIG. 3 illustrates the HPLC data and FIG. 4 illustrates the NMR data.

[Analysis of Compounds HV and HVG]

### Example 3

A high performance liquid chromatography mass spectrometer, LCMS-2010, manufactured by Shimadzu Corporation was used to perform analyses under the following conditions of high performance liquid chromatography and mass spectrometry.

HPLC Conditions

HPLC System: Shimadzu 30A system, Shimadzu Corporation

Analysis column: HITACHI Lachrom Ultra C18 (2.0 mm×50 mm, 2 μm, Hitachi High-Technologies Corporation)

Mobile phase A: 0.1% formic acid solution

Mobile phase B: acetonitrile

Needle washing liquid: acetonitrile

Gradient of Mobile phase A and Mobile phase B was performed according to a time program.

Time program: gradient (performed at the following volume ratio)

TABLE 1

| Time (min) | Mobile phase A (%) | Mobile phase B (%) |
|---|---|---|
| 0.00 | 80 | 20 |
| 3.00 | 80 | 20 |
| 7.50 | 40 | 60 |
| 7.51 | 80 | 20 |
| 10.00 | 80 | 20 |

Flow rate: 0.2 mL/min

Column thermostat setting temperature: 40° C.

Auto-sampler setting temperature: room temperature (no setting)

Injection volume: 10 μL

MS/MS System introduction time: 2.00 minutes to 7.00 minutes

MS/MS conditions

MS/MS System: API 6500 (AB SCIEX)

Ion Source: ESI

Scan Type: MRM

Polarity: Positive

Source Temperature: 600° C.

TABLE 2
Monitored ion:

| Compound | Q1 (m/z) | Q3 (m/z) |
|---|---|---|
| HVG | 727.4 | 532.3 |
| HV | 565.4 | 370.2 |
| Verapamil | 455.4 | 165.2 |

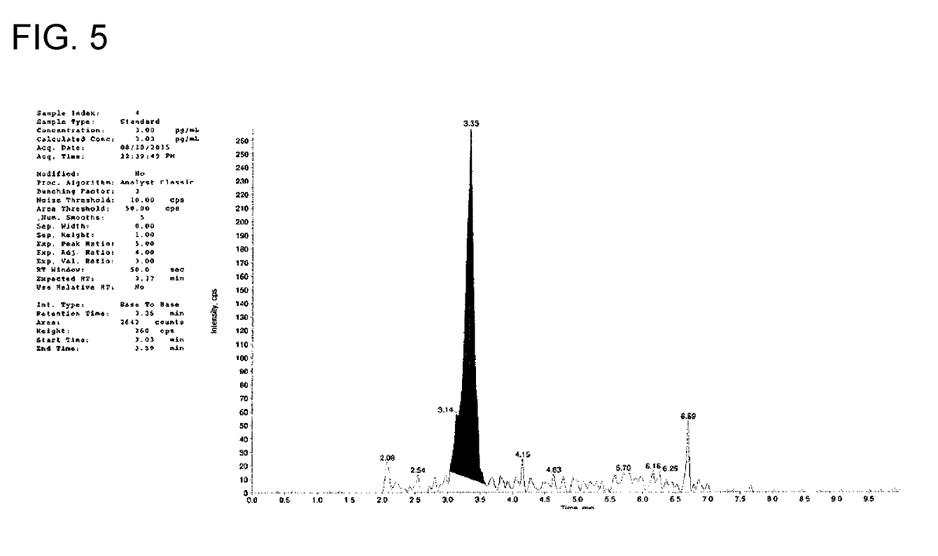

FIG. 5 illustrates analytical results of 3.0 pg/mL of a HVG sample.

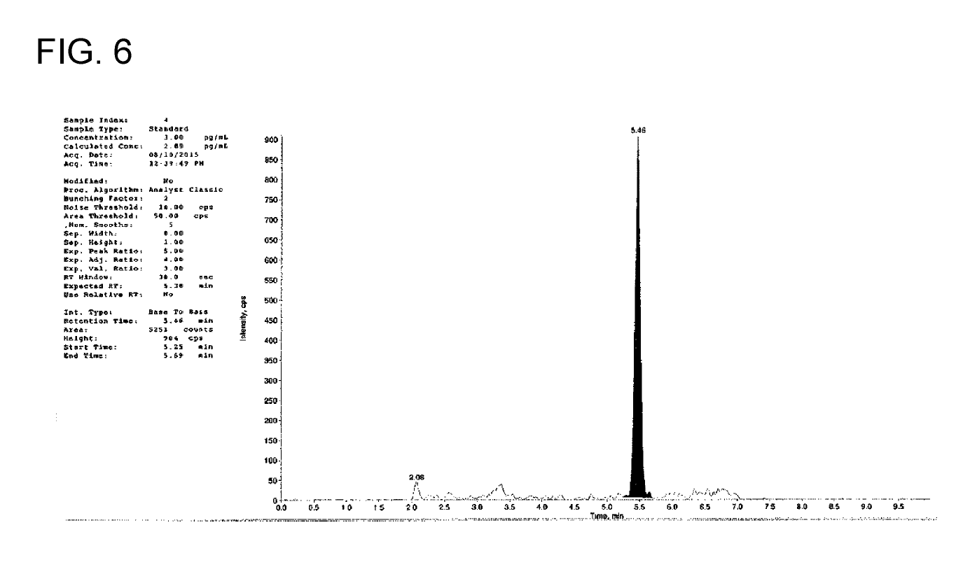

FIG. 6 illustrates analytical results of 3.0 pg/mL of a HV sample.

LOD (S/N=3) was calculated from the analytical results on the basis of the general notices of JIS K0136 (2015) high performance liquid chromatography mass spectrometry. Thus, 4.0 amol for HVG, 1.7 amol for HV, and 2.6 amol for Verapamil were obtained. It was found from this experiment that HV has the same level of sensitivity as Verapamil which is known as a substance which can be detected with a high sensitivity by a mass spectrometer.

[Regarding Enzymatic Reaction]

### Example 4

When a solution of 20 ng/ml of HVG and 0.5 ng/ml of galactosidase in a 10 mmol/L phosphate buffer (pH 7.3) containing 5% (v/v) of methanol and 1 mmol of magnesium chloride was prepared and was reacted at 37° C. for 1 hour, 0.532 ng/ml of HV was then produced. When p-Nitrophenyl galactoside was reacted as a substrate in place of HVG, 0.532 ng/ml of p-nitrophenol was then produced. It was found from this example that HVG has the same level of enzymatic susceptibility as p-nitrophenylgalactoside which is known as a good substrate for galactosidases.

1. A compound represented by the general formula (1): 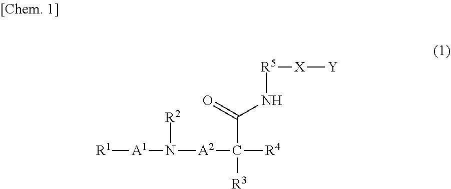
or the general formula (2): 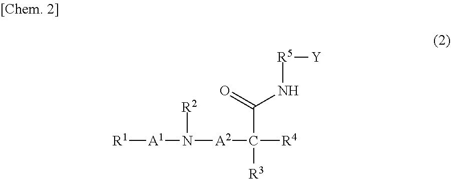
or the general formula (3): 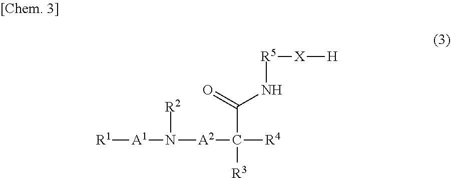
or the general formula (4): 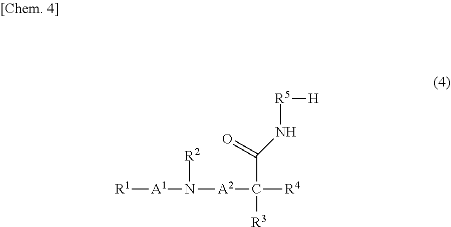
wherein in the general formulae (1), (2), (3), and (4),
R¹, R², R³, and R⁴ may be the same as or different from each other and each represents an alkyl group, an aryl group, a cycloalkyl group, or a heterocyclic group having no substituent or having a substituent W, wherein W represents a C1 to 10 saturated or unsaturated hydrocarbon group, an aryl group, a heterocyclyl group, an alkoxy group, a fluoroalkyl group, an acyl group, an ester group, a hydroxyl group, an amino group, an amide group, a carboxyl group, a sulfonyl group, a nitro group, a cyano group, a sulfenyl group, a sulfo group, a mercapto group, a silyl group, or a halogen group,
R5 represents an aryl group, a cycloalkyl group, or a heterocyclic group not having a substituent other than an —X—Y group, a —Y group, and an —X—H group or having a substituent W other than an —X—Y group, a —Y group, and an —X—H group,
A¹ and A² may be the same as or different from each other and each represents an alkyl group having no substituent or having the substituent W, and
X represents a sulfur atom or an oxygen atom, and
wherein the general formulae (1) and (2), Y is a saccharide,
or a salt thereof.

2. The compound according to claim 1, wherein R⁵ in the general formulae (1) to (4) is a phenyl group, or a salt thereof.

3. The compound according to claim 1, wherein Y in the general formula (1) or (2) is galactose, or a salt thereof.

4. The compound according to claim 1, wherein X in the general formulae (1) and (3) is an oxygen atom, and wherein the compound has an octanol/water partition coefficient, Log P, of 1 to 5 and has a molecular weight of 100 to 1000, or a salt thereof.

5. The compound according to claim 1, wherein the general formula (1) or (2) is the general formula (5): 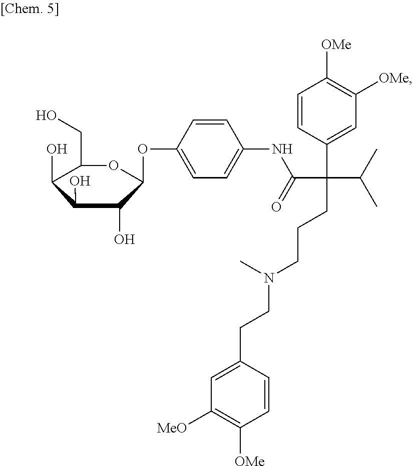
or a salt thereof.

6. The compound or the salt thereof according to claim 1, wherein the general formula (3) or (4) is the general formula (6): 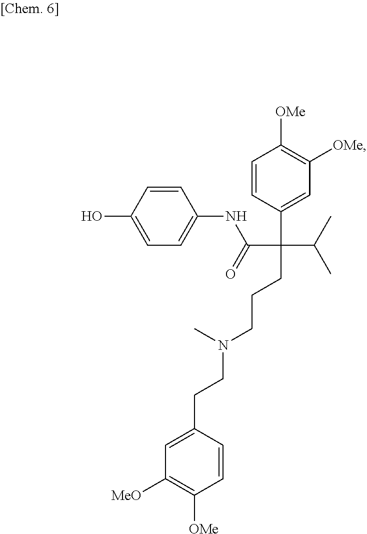
or a salt thereof.

7. A mass spectrometry method comprising a step of reacting an enzyme with the compound of the general formula (1) or (2) according to claim 1 to obtain the compound of the general formula (3) or (4) according to claim 1.

8. The mass spectrometry method according to claim 7, wherein the enzyme is a glycosidase.

9. The mass spectrometry method according to claim 7, wherein the enzyme is a galactosidase.
